(19) United States
(12) Patent Application Publication
(10) Pub. No.: US 2014/0242661 A1
(43) Pub. Date: Aug. 28, 2014

(54) PROCESS CONTROL METHOD

(71) Applicants: Lee Richard Walker, High Wycombe (AU); Sean Neil Sciberras, Sorrento (AU)

(72) Inventors: Lee Richard Walker, High Wycombe (AU); Sean Neil Sciberras, Sorrento (AU)

(73) Assignee: ANAECO LIMITED, Bentley (AU)

(21) Appl. No.: 14/342,127

(22) PCT Filed: Sep. 6, 2012
(86) PCT No.: PCT/AU2012/001057

§ 371 Date: May 15, 2014

(30) Foreign Application Priority Data
Sep. 6, 2011 (AU) 2011903625

(51) Int. Cl. C12P 5/02

(52) U.S. Cl. CPC C12P 5/023
USPC 435/167; 435/290.4; 435/286.5

(57) ABSTRACT

A process control method, the method comprising the steps of: (i) Passing a portion of a biomass containing liquid from a first reactor (12) in which a batch anaerobic digestion of organic material is being or has been conducted to a liquid storage vessel (14), methane being produced in both the first reactor (12) and the liquid storage vessel (14); (ii) Passing a portion of the liquid from the liquid storage vessel (14) back to a first reactor (12) that may or may not be the same reactor (12) of step (i); (iii) Passing a portion of the liquid from either or both of the first reactor (12) and the liquid storage vessel (14) to a second reactor (16); and (iv) Passing a portion of the liquid from the second reactor (16) to either the liquid storage vessel (14) or to the first reactor (12), wherein the direction of liquid to the second reactor (16) allows control of the level of volatile fatty acids (VFA) in the liquid to be returned to the first reactor (12) by way of exposure to additional anaerobic methanogenic microorganisms provided in that second reactor (16). An apparatus (10) for the control of process conditions in a process for the microbiological digestion of organic waste material is also disclosed.

### FIELD OF THE INVENTION

The present invention relates to a process control method. More particularly, the method of the present invention is intended for the optimisation of the performance of the process.

The method of the present invention is understood to have particular application in the control of anaerobic processes for the production of biogases. Further, the method of the present invention is understood to have particular application in the control of anaerobic processes for the production of biogases when those processes are conducted as batch processes.

### BACKGROUND ART

It is known that solid organic waste material may be treated under either anaerobic or aerobic conditions to produce a bioactive, stable end product that, for example, may be used as compost for gardens. This process is achieved through the action of, respectively, anaerobic or aerobic microorganisms that are able to metabolise the waste material to produce the bioactive, stable end product.

It is also, known that the aerobic decomposition of solid organic waste material takes place in the presence of oxygen. The temperature of the waste material rises as some of the energy produced during aerobic decomposition is released as heat, often reaching temperatures of approximately 7520 C. under ambient conditions. The solid end product is often rich in nitrates which are a readily bio-available source of nitrogen for plants, making the end product particularly suitable as a fertiliser.

It is further known that the anaerobic digestion of solid organic waste material takes place in the absence of oxygen. Anaerobic microbial metabolism is understood to be optimised when the organic material is heated to temperatures at which mesophilic or thermophilic bacteria are operative. The process of anaerobic microbial metabolism results in the production of biogas, in turn predominantly methane and carbon dioxide. The solid product of the process is often rich in ammonium salts. Such ammonium salts are not readily bio-available and are, consequently, generally treated under conditions in which aerobic decomposition will occur. In, this manner the material is used to produce a product that is bio-available.

Typically, systems for the biodegradation of organic waste material are directed to either aerobic or anaerobic processes. However, there are a small number of systems that have sought to combine both anaerobic and aerobic biodegradation processes. The processes of German Patent 4440750 and International Patent Application PCT/DE1994/000440 (WO 1994/024071) each describe the combination of an anaerobic fermentation unit and an aerobic composting unit. Importantly, these systems describe discrete and separate vessels for the aerobic and anaerobic biodegradation processes. One consequence of this arrangement is that such processes are able to be conducted on a continuous, as opposed to a batch, basis. Continuous processes such as these, whilst they have drawbacks in terms of the potential for short-circuiting, which has a negative impact on pasteurisation of organics, do allow continuous control of ‘feed’ for the microorganisms present.

International Patent Application PCT/AU00/00865 (WO 01/05729) describes an improved process and apparatus in which many of the inefficiencies of the previous processes and apparatus are overcome. The improved process and apparatus are characterised at a fundamental level by the sequential treatment of organic waste material in a single vessel, through an initial aerobic step to raise the temperature of the organic waste material, an anaerobic digestion step and a subsequent aerobic treatment step. During the anaerobic digestion step a process water or an inoculum containing microorganisms is introduced to the vessel to create conditions suitable for efficient anaerobic digestion of the contents and the production of biogas. The introduced inoculum also aids in heat and mass transfer as well as providing buffer capacity to protect against acidification. Subsequently, air is introduced to the residues in the vessel to create conditions for aerobic degradation. It is further described that the water introduced during anaerobic digestion may be sourced from an interconnected vessel that has undergone anaerobic digestion.

The sequential treatment of organic waste material in a single vessel requires that the process be conducted as a batch process. Whilst the single vessel process described in International Patent Application PCT/AU00/00865 (WO 01/05729) provides many advantages with respect to prior art processes, it does create challenges in maintaining process stability during anaerobic treatment. Amongst these is an inability to control the rate of organic acid generation during early anaerobic digestion.

The microorganisms employed during anaerobic digestion of the biomass typically comprise a delicate balance of acid producing and acid consuming micro-organisms. For example, in an uninoculated system the number of acid producing micro-organisms typically exceed the number of acid consuming micro-organisms.

Acid producing bacterial, species will produce organic acids which will typically cause the pH of a decomposing biomass to drop (become more acidic). Acid consuming microbial species contribute to the production of biogas, including methane, and cause the pH to rise (become more alkaline or basic). Early in a typically batch anaerobic digestion, the number of organic acid producing bacteria exceed those that consume these acids. This imbalance can result in acidification, process instability and/or process failure and highlights the need for accurate monitoring of the process.

Similarly, the introduction of microorganisms to the reactor is not something that can readily be monitored when the process is being conducted on a commercial scale and in real time. In the process of International Patent Application PCT/AU00/00865 (WO 01/05729) the liquid produced during the anaerobic phase of decomposition is re-used. As such, the process is re-exposed to what has been produced in that earlier anaerobic phase and is present in the liquid that has been re-used. Consequently, the conditions in the reactor may become too acidic over time. This is particularly the case if the level of volatile fatty acids (VFAs) is rising due to incomplete microbial exhaustion of the VFA present in the liquid from a previous batch prior to reintroduction to the reactor. The postulated decrease in pH may eventually lead to process failure.

Similarly, temperature maintenance of static high solids batch anaerobic digestion processes becomes difficult due to poor mixing and inefficient mass transfer. The ensuing unfavourable conditions may also provide poor microbial performance, such as a decrease in the metabolism of the microorganisms as a result of lower temperature. In turn, the performance of the degradation process and the production of biogas are hampered.

In Cha et al., “Characteristics of temperature change on the substrate degradation and bacterial population in one-phase and two-phase anaerobic digestion’, Environ. Eng. Res. 2001, vol. 6, pages 99-108 there is disclosed a continuously fed mesophilic, wet, 2-stage anaerobic digestion system. An initial feed is introduced to a first reactor (having a pH of between 5.5 and 5.8) where hydrolysis and acidogenic reactions occurred. The effluent from the first reactor was introduced to the second reactor where methane production occurred. Chemostat-type reactors were utilised, suggesting that liquid of a constant composition was added to, and withdrawn from, the reactor at constant rate. The performance of the first reactor is optimised to provide a feed for the second reactor in which methane is produced. This requires a separate biology in each reactor. As such, this arrangement is not a suitable solution for a system in which effluent from one acid producing reactor is removed and subsequently returned to that same reactor.

In Kraemer et al., “Continuous fermentative hydrogen production using a two-phase reactor system with recycle”, Environ. Sci. Technol. 2005, vol. 39, pages 3819-3825, there is disclosed a continuously fed mesophilic, wet, 2-stage anaerobic digestion system. A first reactor is optimised for H₂ gas production and is operated as “a chemostat”. In effect this means that liquid of a constant composition was added to, and withdrawn from, the first reactor at a constant rate. A feed is introduced to the first reactor (at pH 5.5) where hydrolysis, acidogenic reactions and H₂ gas production occurs. An effluent from this first reactor is introduced to a second reactor in which methane production occurs. A portion of the effluent from the second reactor was recycled back to the first reactor although this was demonstrated to be detrimental to H₂ gas production. Again, the first reactor is optimised to produce H₂ gas and an acidic solution to be consumed in the second reactor for the production of methane. As such, the biology in the two reactors is different. As such, this arrangement is again not a suitable solution for a system in which effluent from one acid producing reactor is removed and subsequently returned to that same reactor.

US Publication US 2007/0158264 A1 discloses a two phase anaerobic digestion system comprising one or more hydrolysis reactors, a buffer tank and a methane producing reactor. The or each hydrolysis reactor generates H₂ gas and an acidic liquor at a pH of between 4.5 and 7, preferably 4.5 to 6, from a slurry of organic feedstock. The buffer tank equilibrates variations in liquor composition prior to feeding to the methane producing reactor. No liquor is fed from this buffer tank to the or each first reactor. A portion of the effluent from the methane producing reactor is recycled back to the or each hydrolysis reactor. The or each hydrolysis reactor is optimised for the production of H₂ gas and an acidic liquor, and contains a suitable bacterial population for doing so. The methane producing reactor is optimised for methane production and contains a suitable bacterial population to achieve this aim. As such, the biology in the two reactors is again different.

The process of the present invention has as one object thereof to overcome substantially the abovementioned problems associated with the prior at, or to at least provide a useful alternative thereto.

The preceding discussion of the background art is intended to facilitate an understanding of the present invention only. The discussion is not an acknowledgement or admission that any of the material referred to is or was part of the common general knowledge as at the priority date of the application.

Throughout the specification and claims, unless the context requires otherwise, the word “comprise” or variations such as “comprises” or “comprising”, will be understood to imply the inclusion of a stated integer or group of integers but not the exclusion of any other integer or group of integers.

### DISCLOSURE OF THE INVENTION

In accordance with the present invention there is provided a process control method, the method comprising the steps of:
(i) Passing a portion of a biomass containing liquid from a first reactor in which a batch anaerobic digestion of organic material is being or has been conducted to a liquid storage vessel, methane being produced in both, the first reactor and the liquid storage vessel;
(ii) Passing a portion of the liquid from the liquid storage vessel back to a first reactor that may or may not be the same reactor of step (i);
(iii) Passing a portion of the liquid from either or both of the first reactor and the liquid storage vessel to a second reactor; and
(iv) Passing a portion of the liquid from the second reactor to either the liquid storage vessel or to the first reactor,
   wherein the direction of liquid to the second reactor allows control of the level of volatile fatty acids (VFA) in liquid to be returned to the first reactor by way of exposure to additional anaerobic methanogenic microorganisms provided in that second reactor.

Preferably, the second reactor is provided in the form of either a low solids wet system or a high solids semi-dry system. Additionally, the second reactor may be a continuously fed reactor.

Still preferably, the measured level of VFA is used to determine at what rate the biomass containing liquid is fed to the second reactor. During periods in which the biomass containing liquid is considered to have high VFA levels, then that liquid is preferably fed slowly to the second reactor. During periods in which the liquid contains what are considered to be low VFA levels then that liquid is fed more quickly to the second reactor. In this manner the amount of VFA fed to the second reactor is maintained generally constant and the biology therein is exposed to the same concentrations of soluble food.

In one form of the present invention the feed rate of liquid to the second reactor is confirmed by consideration of the levels of VFA in the effluent of the second reactor. In a further form of the present invention the feed rate to the second reactor is confirmed by way of a comparison of the VFA concentration of the feed to the second reactor and the methane generation rate therefrom.

Effluent from the second reactor is preferably discharged at the same rate as liquid is fed to it.

Preferably, more than a single batch operated first reactor is provided whereby feed to the second reactor may be maintained as continuous.

The maintenance of low VFA levels in the liquid to be returned to the first reactor in the above manner allows any decrease in pH therein to be minimised.

In a further form of the present invention the temperature of the biomass containing liquid is also measured externally to the first reactor. Should the measured temperature not be considered appropriate then the biomass containing liquid is preferably directed to one or more heat exchangers by which the temperature of the biomass containing liquid may be increased prior to reintroduction thereof to the first reactor.

In accordance with the present invention there is further provided an apparatus for the control of process conditions in a process for the microbiological digestion of organic waste material, the apparatus comprising at least two first reactors in which the organic waste material may be located, a storage vessel for biomass containing liquid and a second reactor, whereby biomass containing liquid is able to be passed between at least one of the first reactors, the storage container and the second reactor.

Preferably, the second reactor is provided in the form of a continuously fed reactor.

The apparatus preferably further comprises control means whereby the rate of feed to the second reactor is able to be varied dependent upon one or more measured characteristics of the biomass containing liquid.

Still preferably, the one or more measured characteristics of the biomass containing liquid are obtained from measures taken in or around the second reactor.

Still further preferably, the apparatus further comprises control means whereby the rate of flow of biomass containing liquid from the first reactor vessel to the second reactor is able to be varied dependent upon one or more measured characteristics of the biomass containing liquid.

In one form of the invention the measures include a measure of VFA content.

Preferably, the measures further include a measure of the temperature of the biomass containing liquid.

### BRIEF DESCRIPTION OF THE DRAWINGS

The present invention will now be described, by way of example only, with reference to one embodiment thereof and the accompanying drawing, in which:

FIG. 1 is a schematic representation of an apparatus for the control of process conditions in a process for the microbiological digestion of organic waste material, the apparatus being in accordance with one embodiment of the present, invention.

### BEST MODE(S) FOR CARRYING OUT THE INVENTION

In International Patent Application PCT/AU00/00865 (WO 01/05729), the entire content of which is incorporated herein by reference, both a process and a reactor, vessel for the anaerobic digestion and aerobic composting of organic waste material are described. The process is described as one in which successive stages of anaerobic digestion and aerobic composting occur in a single reactor vessel. The anaerobic stage of the process involves the introduction of liquid to the reactor to inoculate the biomass with microorganisms from a previous batch cycle to facilitate and accelerate the initial production of biogas. The biomass containing liquid is subsequently removed from the reactor to allow the aerobic stage of the process to proceed. The removed biomass containing liquid is temporarily stored before being reintroduced to the reactor at the next anaerobic stage, or it may alternatively pumped directly into the reactor next in line in the batch sequence.

In accordance with the present invention there is provided a process control method, the method comprising the steps of:
(i) Passing a portion of a biomass containing liquid from a first reactor in which a batch anaerobic digestion of organic material is being or has been conducted to a liquid storage vessel, methane being produced in both the first reactor and the liquid storage vessel;
(ii) Passing a portion of the liquid from the liquid storage vessel back to a first reactor that may or may not be the same reactor vessel of step (i);
(iii) Passing a portion of the liquid from either or both of the first reactor and the liquid storage vessel to a second reactor; and
(iv) Passing a portion of the liquid from the second reactor to either the liquid storage vessel or to the first reactor,
   wherein the direction of liquid to the second reactor allows control of the level of volatile fatty acids (VFA) in liquid to be returned to the first reactor by way of exposure to additional anaerobic methanogenic microorganisms provided in that second reactor.

In one form of the present invention the second reactor is provided in the form of a continuously fed wet or low solids anaerobic digestion system or reactor having a solids content less than 20% dry matter. It is to be understood that the second reactor may alternatively be provided in the form of a continuously fed high solids reactor having a solids content greater than 20% dry matter.

The measured level of VFA is used to determine at what rate the biomass containing liquid is fed to the second reactor. During periods in which the biomass containing liquid is considered to have high VFA levels, then that liquid is preferably fed slowly to the second reactor. During periods in which the liquid contains what are considered to be low VFA levels then that liquid is fed more quickly to the second reactor. In this manner the amount of VFA fed to the second reactor is maintained generally constant and the biology therein is exposed to the same concentrations of soluble food.

The feed rate of liquid to the second reactor is confirmed by consideration of the levels of VFA in the effluent of the second reactor. Effluent withdrawn from the second reactor is preferably at the same rate as liquid is fed to it. The feed rate to the second reactor may further be confirmed by way of a comparison of the VFA concentration of the feed to the second reactor and the methane generation rate therefrom.

More than a single batch operated first reactor is provided whereby feed to the second reactor may be maintained as continuous.

The maintenance of low VFA levels in the liquid to be returned to the first reactor in the above manner allows any decrease in pH therein to be minimised.

The temperature of the biomass containing liquid is also measured externally to the first reactor. Should the measured temperature not be considered appropriate then the biomass containing liquid is preferably directed to one or more heat exchangers by which the temperature of the biomass containing liquid may be increased prior to reintroduction thereof to the first reactor.

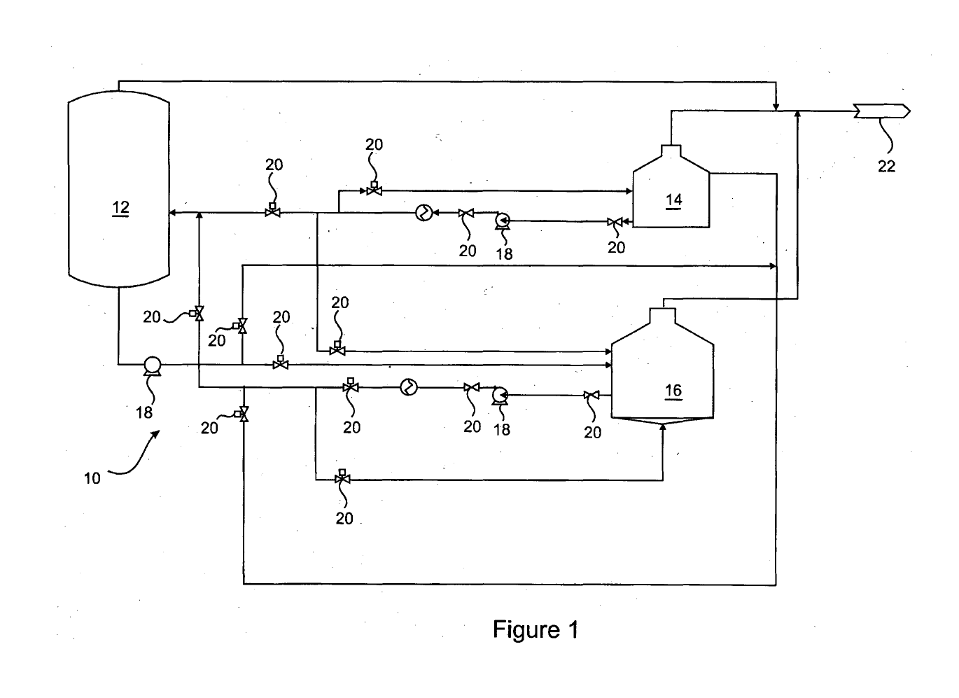

In FIG. 1 there is shown an apparatus 10 for the control of process conditions in a process for the microbiological digestion of organic waste material, the apparatus comprising at least two first digesters or reactors 12 (only one shown) in which the organic waste material may be located, a storage vessel 14 for biomass containing liquid and a second reactor, provided in the form of a low solids wet system, for example a continuously fed reactor (CFR) 16, whereby biomass containing liquid is able to be passed between the first reactor 12, the storage vessel 14 and the CFR 16.

The apparatus 10 further comprises control means whereby the rate of flow of the biomass containing liquid from the storage vessel 14 to the reactor 12 is able to be varied dependent upon one or more measured characteristics of the biomass containing liquid. The control means comprising at least in part a series of pumps 18 and valves 20 provided in the various transfer lines connecting the first reactor 12, the storage vessel 14 and the CFR 16, as shown in FIG. 1.

The one or more measured characteristics of the biomass containing liquid are obtained from measures taken in or around the CFR 16. The measures include a measure of VFA content and a measure of the temperature of the biomass containing liquid. The apparatus 10 further comprises control means whereby the rate of flow of biomass containing liquid from the reactor 12 to the CFR 16 is able to be varied dependent upon one or more measured characteristics of the biomass containing liquid.

As shown in FIG. 1, biomass containing liquid may be directed to the CFR 16 from the reactor 12 or from the storage vessel 14. Additionally, biomass containing liquid may be directed from the CFR 16 to the reactor 12 or to the storage vessel 14, as the needs of the process require. Further, the rate of transfer is able to be controlled, as described above.

The CFR 16 is in one form of the present invention provided in the form of a continuously stirred tank reactor.

As described in International Patent Application PCT/AU00/00865 (WO 01/05729), once the organic waste decomposition reactor vessel has been loaded with organic waste, anaerobic liquor/inoculum is introduced. The organic fraction of municipal solid waste (“OFMSW”) loaded into the vessel contains both VFA producing and VFA consuming micro-organisms. The number of microorganisms that produce VFA typically exceeds those that consume them. The purpose of the anaerobic liquor addition is three-fold, first being to increase the number of VFA consumers and secondly to provide a buffer to minimise the depression in the pH of the liquor should VFA generation exceed consumption. The experience of the Applicant is such that during the first 2-3 days of anaerobic treatment, VFA production exceeds consumption and the pH of the anaerobic liquor falls. A third purpose of the anaerobic liquor addition is to improve mass and energy transfer.

The liquor containing soluble organics and VFA, including propionate, from the reactor 12 is fed, at a controlled rate, to the CFR 16, in which the organics and VFA are converted into biogas 22. The liquor addition rate is set such that the effluent from the CFR 16 will contain low levels of VFA prior to it being reintroduced to the reactor vessel, typically less than 1 mM. Monitoring of the CFR 16 effluent is performed on-line so that the liquor flow rate from the reactor vessel to the CFR 16 is controlled by a distributed control system (“DCS”). At times when the organic load of the liquor within the reactor 12 is high, the rate of addition to the CFR 16 is reduced. Conversely, at times when the quantity of soluble organics within the liquor is low (i.e. towards the completion of digestion) the liquor exchange from the reactor vessel to the CFR 16 is higher. The amount of liquor added to the CFR 16 and that withdrawn is matched so that the CFR 16 liquor volume remains substantially constant.

If the CFR 16 is fed relatively low levels of VFA as ‘food’ it is understood that the levels of propionate present will be decreased more quickly when compared with the addition of higher levels of VFA. Thermodynamically, propionate is understood to be the most difficult VFA for anaerobic microorganisms to degrade. An elevated concentration of VFA, in particular, acetate, a common end-product of anaerobic microbial degradation of organic compounds, makes the anaerobic degradation of propionate thermodynamically less favourable. Consequently, the maintenance of continuously low levels of VFA within the CFR 16 ensures that propionate degradation is thermodynamically favourable and that propionate can be continuously degraded. It is the experience of the Applicants that either the on-going or periodic addition of high levels of VFA to the CFR 16 does not reduce propionate levels in an equivalent manner.

Feed rates to the CFR 16 will to a large extent be dependent upon the capacity of the CFR 16. To allow for the appropriate dilution, a larger reactor will provide greater dilution and as a consequence a greater feed rate. Feed rates to the CFR 16 are also a function of the dilution rate with higher flows providing a shorter retention time and a greater propensity to wash the biology out of the CFR 16. Understanding this, feed rate to the CFR 16 may be between about 2 and 5 m³/h, for example about 3.5 m³/h.

In the Applicant's experience, typical VFA concentration in the liquid being fed to the CFR 16 is between about 100 and 300 mM. Approximate average concentrations of each of acetate, propionate and butyrate are about 140, 40 and 100 mM, respectively. High and low levels of each of acetate, propionate and butyrate are, for example, 180, 30, 140 mM, and 65, 15, 20 mM, respectively.

Monitoring of VFA within the reactor 12 provides an additional control parameter. The appropriate flow rate to the CFR 16 can be predicted based on the VFA concentration of the liquor within the reactor 12 and a desired VFA concentration within the CFR 16. The predicted and actual flow rates can also be compared to provide a measure of CFR health/efficiency. The feed rate to the second reactor may further be confirmed by way of a comparison of the VFA concentration of the feed to the second reactor and the methane generation rate therefrom. Knowing the VFA concentration and the feed rate, a theoretical methane generation rate can be established. Should the actual methane generation rate be lower than the theoretical, then VFA will accumulate in the CFR 16. This can be confirmed via the concentration of VFA in the effluent of the CFR 16 and the feed rate can be adjusted accordingly.

Acid consuming reactions (i.e. methane producing reactions) also occur within the reactor 12. During the initial stages of digestion, methane production is typically low due to the small numbers of methane producing micro-organisms present in the reactor 12. However, the rate of methane production within the reactor 12 is expected to increase toward the end of each batch due to microbial growth therein. For efficient growth of acid consuming biomass (methane producers) within the reactor 12, pH must be controlled. Buffer addition along with careful selection of recirculation pathways of the liquor within the reactor 12 may be required to avoid acidification of the reactor contents and to maintain stable process operation.

It is envisaged that a well maintained CFR 16 will be of significant advantage to operation of the reactor described in International Patent Application PCT/AU00/00865 (WO 01/05729), as acidity, in the form of VFA, can be reduced. Consequently, as the effluent from the CFR is recycled back to the reactor, the liquor flow between the reactor and the CFR not only transports soluble organics to the CFR (reducing acidity therein) but also dilutes the VFA contained within the anaerobic liquor in the reactor, enabling greater process stability. The typical depression in the pH, during the early part of anaerobic digestion, is reduced in this manner.

It can be seen with reference to the above description that each of the vessels, including the reactor 12, the vessel 14 and the CFR 16, are optimised to produce methane. Consequently, the biology in each of these vessels is essentially the same.

Modifications and variations such as would be apparent to the skilled addressee are considered to fall within the scope of the present invention.

1. A process control method, the method comprising the steps of:
(i) Passing a portion of a biomass containing liquid from a first reactor in which a batch anaerobic digestion of organic material is being or has been conducted to a liquid storage vessel, methane being produced in both the first reactor and the liquid storage vessel;
(ii) Passing a portion of the liquid from the liquid storage vessel back to a first reactor that may or may not be the same reactor of step (i);
(iii) Passing a portion of the liquid from either or both of the first reactor and the liquid storage vessel to a second reactor; and
(iv) Passing a portion of the liquid from the second reactor to either the liquid storage vessel or to the first reactor,
wherein the direction of liquid to the second reactor allows control of the level of volatile fatty acids (VFA) in liquid to be returned to the first reactor by way of exposure to additional anaerobic methanogenic microorganisms provided in that second reactor.

2. A method according to claim 1, wherein the second reactor is provided in the form of either a low solids wet system or a high solids semi-dry system.

3. A method according to claim 1, wherein the measured level of VFA is used to determine at what rate the biomass containing liquid is fed to the second reactor.

4. A method according to claim 1, wherein if the biomass containing liquid is considered to have high VFA levels, then that liquid is fed slowly to the second reactor.

5. A method according to claim 1, wherein if the liquid contains what are considered to be low VFA levels then that liquid is fed more quickly to the second reactor.

6. A method according to claim 1, wherein the amount of VFA fed to the second reactor is maintained generally constant and the biology therein is exposed to the same concentrations of soluble food.

7. A method according to claim 3, wherein the feed rate of liquid to the second reactor is confirmed by consideration of the levels of VFA in the effluent of the second reactor.

8. A method according to claim 3, wherein the feed rate to the second reactor is confirmed by way of a comparison of the VFA concentration of the feed to the second reactor and the methane generation rate therefrom.

9. A method according to claim 1, wherein effluent from the second reactor is at substantially the same rate as liquid is fed to it.

10. A method according to claim 1, wherein more than a single batch operated first reactor is provided whereby feed to the second reactor may be maintained as continuous.

11. A method according to claim 1, wherein the maintenance of low VFA levels in the liquid to be returned to the first reactor allows any decrease in pH therein to be minimised.

12. A method according to claim 1, wherein the temperature of the biomass containing liquid is also measured externally to the first reactor.

13. A method according to claim 12, wherein should the measured temperature not be considered appropriate then the biomass containing liquid is directed to one or more heat exchangers by which the temperature of the biomass containing liquid may be increased prior to reintroduction thereof to the first reactor.

14. An apparatus for the control of process conditions in a process for the microbiological digestion of organic waste material, the apparatus comprising at least two first reactors in which the organic waste material may be located, at least one storage vessel for biomass containing liquid and a second reactor, whereby biomass containing liquid is able to be passed directly between each of at least one of the first reactors, at least one storage container and the second reactor.

15. An apparatus according to claim 14, wherein the second reactor is provided in the form of either a low solids wet system or a high solids semi-dry system.

16. An apparatus according to claim 14, wherein the apparatus further comprises control means whereby the rate of feed to the second reactor is able to be varied dependent upon one or more measured characteristics of the biomass containing liquid.

17. An apparatus according to claim 16, wherein the control means facilitates confirmation of the feed rate to the second reactor by way of a comparison of a VFA concentration of the feed to the second reactor and a methane generation rate therefrom.

18. An apparatus according to claim 16, wherein the one or more measured characteristics of the biomass containing liquid are obtained from measures taken in or around the second reactor.

19. An apparatus according to claim 1, wherein the apparatus further comprises control means whereby the rate of flow of biomass containing liquid from the first reactor vessel to the second reactor is able to be varied dependent upon one or more measured characteristics of the biomass containing liquid.

20. An apparatus according to claim 16, wherein the measures include a measure of VFA content.

21. An apparatus according to claim 16, wherein the control means is capable of measuring the temperature of the biomass containing liquid.

22. (canceled)

23. (canceled)
